(19) United States
(12) Patent Application Publication
(10) Pub. No.: US 2024/0279150 A1
(43) Pub. Date: Aug. 22, 2024

(54) PROCESS FOR THE MANUFACTURE OF CARBOXYLIC ANHYDRIDE

(71) Applicant: Cerdia International GmbH, Basel (CH)

(72) Inventors: Dirk Hölter, Emmendingen (DE); Andreas Hummel, Freiburg (DE)

(73) Assignee: Cerdia International GmbH, Basel (CH)

(21) Appl. No.: 18/563,005

(22) PCT Filed: May 11, 2022
(86) PCT No.: PCT/EP2022/062741

§ 371 Date: Nov. 21, 2023

(30) Foreign Application Priority Data
May 21, 2021 (MY) PI2021002821

(51) Int. Cl. C07C 51/56; B27K 3/36; C08B 3/06

(52) U.S. Cl. CPC C07C 51/56; B27K 3/36; C08B 3/06

(57) ABSTRACT

The present invention concerns a process for manufacturing a carboxylic anhydride by reaction of a carboxylic acid with a ketene, and the use of the thus prepared carboxylic anhydride in a process for acylation of polysaccharides.

The present invention concerns a process for the production of a carboxylic anhydride by reacting a carboxylic acid with a ketene, and the use of the carboxylic anhydride, which is thus produced, in an acylation process of polysaccharides.

Carboxylic anhydride is commonly used as acylation agent of polysaccharides as well as polysaccharide-containing substances such as wood. Acylated, and in particular acetylated, wood is frequently used e.g. as construction material where it provides a high service life and excellent resistance against environmental degradation and pathogens. An example of a conventional wood acetylation process is described, for example, in EP 680810 whose relevant contents are incorporated by reference into the present patent application.

As polysaccharides in wood are acylated with a carboxylic anhydride, carboxylic acid (AcylOH) is formed:

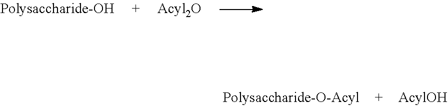

Thus, carboxylic acid is the major by-product of wood acetylation, the disposal of which is a relevant cost factor. The problem with this carboxylic acid is its contamination with hydrocarbons and other organic and inorganic impurities, which become entrained with the acid during the wood acylation reaction. For example, chlorine can be eluted from the wood, which on thermal processing with the acid may form acyl chlorides having an increased corrosiveness. Another type of frequent impurities in carboxylic acids derived from wood acylation are terpenes, which might interact with reactions for which the carboxylic acid is subsequently used. Thus, waste carboxylic acid from wood acylation processes can only be used further in processes, wherein such impurities are not critical.

An example of such process is provided in US 2009/0275777, which describes the further use of wood acylation waste acid as a solvent for the oxidation of xylene to provide phthalic acid. In this process, terpene impurities in the acid solvent are oxidized to carbon dioxide. US 2009/0275777 further schematically describes the combination of a “recycled” acid with fresh acetic acid, which is used as a feed stream for a cracking vessel to form ketene.

Due to the acid being contaminated with impurities, the price obtainable for such acid on the market is low. In addition, since about half of the carboxylic acid used in the wood acetylation is lost, the process is not highly economical either.

A means to address this problem is to purify the carboxylic acid by-product to thus enable a regeneration thereof to carboxylic anhydride. Such regeneration is e.g. possible by reacting the carboxylic acid with a ketene (as a dehydrated form of the acid). To be economically feasible however, it has to be made sure that the regeneration of the carboxylic anhydride is less expensive than the disposal of the acid. Here, the more effective the carboxylic anhydride can be regenerated, the more efficient the process of polysaccharide and in particular wood acylation will be in the end.

As noted above, by-product carboxylic acid from wood acetylation processes regularly contains impurities, which are eluted from the wood during acylation thereof. A relevant type of such impurities are aldehydes and ketones, which are formed in the wood via oxidative degradation of unsaturated fatty acids. In an acidic environment, such aldehydes and ketones will react with ketene to form enolacetates and provides unwanted by-products which may be even more difficult to separate from the carboxylic anhydride than the aldehydes and ketones. Moreover, there is a risk that aldehydes and ketones are degraded under the harsh conditions, where the ketene is formed, leading to soiling of the ketene production unit.

To remove these impurities, the carboxylic acid containing wood treatment fluid is thus regularly subjected to various processing steps, where the impurities are removed from the carboxylic acid. As these steps require significant input of energy, it is clear that such purification increases overall costs and reduces the economic feasibility of the process.

In order to enhance the efficiency and overall economics of acylation processes such as the aforesaid wood treatment process, the present invention proposes an economically efficient process for the preparation of carboxylic anhydride from polysaccharide and in particular wood acylation fluids.

The present invention in one aspect concerns a process for the production of carboxylic anhydride, and preferably acetic anhydride, wherein a carboxylic acid, preferably acetic acid, is reacted with a ketene to produce the carboxylic anhydride, wherein
I. the ketene is introduced into a carboxylic acid comprising C₂₋₁₂ aldehydes and/or C₃₋₁₂ ketones to thus form carboxylic anhydride and/or
II. at least part of the ketene is manufactured from a carboxylic acid comprising C₂₋₁₂ aldehydes and/or C₃₋₁₂ ketones, and the thus produced ketene is reacted with a carboxylic acid to thus form carboxylic anhydride.

For condition I., the content of C₂₋₁₂ aldehydes and/or C₃₋₁₂ ketones is preferably in the range of from 5 to 3000 ppm, more particularly of from 5 to 1500 ppm, by weight and/or for condition II., the content of C₂₋₁₂ aldehydes and/or C₃₋₁₂ ketones is preferably equal to or less than 3000 ppm, more particularly 1500 ppm, by weight of. In a particularly preferred embodiment of condition II., the content of C₂₋₁₂ aldehydes and/or C₃₋₁₂ ketones is in the range of from 10 to 100 ppm by weight. When in the above, the content of C₂₋₁₂ aldehydes and/or C₃₋₁₂ ketones is equal to or less than 3000 ppm, this mean that the C₂₋₁₂ aldehydes and/or C₃₋₁₂ ketones must still be present in the composition, which is subjected to a ketene generation process. Preferably, the content of such aldehydes and ketones is at least 1 ppm and in particular at least 5 ppm. In the context of the invention, the content of C₂₋₁₂ aldehydes and/or C₃₋₁₂ ketones is the combined amount of the respective aldehydes and ketones in ppm.

In a further aspect, the invention concerns a process for the manufacture of acylated wood, and in particular acetylated wood, wherein the carboxylic anhydride used therein is produced according to a process as described above and the wood is treated with the carboxylic anhydride thus prepared. A yet further aspect of the invention concerns the use of carboxylic anhydride, which has been manufactured according to the above-mentioned processes, for the acylation of polysaccharides. In a preferred embodiment of this use, the polysaccharides, which are acylated, are polysaccharides in wood.

It has been found that surprisingly, a carboxylic acid comprising C₂₋₁₂ aldehydes and/or C₃₋₁₂ ketones may be used “as is” or with only minimal purification for the preparation of carboxylic anhydride when the carboxylic anhydride, preferably acetic anhydride, is manufactured via the ketene route. In spite of the presence of the aldehyde and ketone impurities, in this case the process is not markedly adversely affected.

Accordingly, it is possible to use carboxylic acid, which is produced as an effluent stream of a polysaccharide acylation process for ketene generation. Thus in a preferred embodiment the carboxylic acid for use in conditions I. and/or II is a carboxylic acid which is produced in a polysaccharide acylation process. In particular, this polysaccharide acylation process is a wood acylation process, and even more particularly a wood acetylation process. In addition, disclosed are corresponding combination processes, wherein in a first step the carboxylic acid is generated by acylating polysaccharides (preferably wood) and in a second step the carboxylic acid is at least in part reprocessed to carboxylic anhydride via a process as described above.

In a first embodiment of the inventive process, where condition I. is met, a carboxylic acid comprising C₂₋₁₂ aldehydes and/or C₃₋₁₂ ketones, preferably in an amount as specified above, is fed to a thermal treatment process, in particular a cracking process, to produce ketene, which can then further be reacted with a carboxylic acid to form carboxylic anhydride. Surprisingly the ketene production proceeds without side-products in unacceptable amounts or nature or unacceptable equipment soiling.

In a second embodiment of the inventive process, where condition II. is met, the carboxylic acid which further comprises C₂₋₁₂ aldehydes and/or C₃₋₁₂ ketones is used as absorption acid, wherein the term “absorption acid” generally denotes the carboxylic acid which is reacted with ketene to provide carboxylic anhydride by the introduction of ketene therein. The obtained carboxylic anhydride is particularly suited for recycling back to the process from which the carboxylic acid and impurities originates, in particular wood treatment processes.

As C₂₋₁₂ aldehydes and/or C₃₋₁₂ ketones are tolerated in the process for the production of carboxylic anhydride, the invention avoids laborious removal of such aldehydes and/or ketones from the carboxylic acid which is used to produce the carboxylic anhydride. On the other hand, the carboxylic acid may be subjected to purification steps, which are not particularly designed for or are aiming at the removal of C₂₋₁₂ aldehydes and/or C₃₋₁₂ ketones. Examples of such steps are filtration or crude evaporation, stripping or distillation steps, which remove solid contaminants. While this is not particularly aimed at, these steps can result in a partial reduction of C₂₋₁₂ aldehydes and/or C₃₋₁₂ ketones in the carboxylic acid.

In the present invention the carboxylic acid, and in particular acetic acid, for use in the inventive process often originates from industrial processes such as, for example, acetylation of polysaccharides such as wood treatment processes. Preferably, the carboxylic acid originates from a wood treatment process, and in particular a wood acetylation process.

In the inventive process, the carboxylic acid is preferably selected from the group of straight or branched C2 to C7 alkyl or alkylene carboxylic acids of the formula R³COOH, wherein R³ is selected from the group consisting of straight or branched C1 to C6 alkyl or alkylene groups which can optionally be substituted, for example by at least one substituent selected from the group of halogens, OH and CN, and/or which can optionally be unsaturated. Preferably, the carboxylic acid is selected from the group consisting of acetic acid, trifluoroacetic acid, propionic acid, n-butyric acid, iso-butyric acid and acrylic acid. Acetic acid is the most preferred carboxylic acid.

In the process according to the invention, the carboxylic acid comprises C₂₋₁₂ aldehydes and/or C₃₋₁₂ ketones. Relevant C₂₋₁₂ aldehydes include unsaturated and saturated aldehydes such as e.g. hexanal. Relevant C₃₋₁₂ ketones include saturated ketones such as acetone, 2-butanone, 3-pentanone, 3-methyl-2-butanone, 4-methyl-2-pentanone and 2-heptanone and unsaturated ketones such as 4-methyl-3-penten-2-one and 3-pentene-2-one. The amount of C₂₋₁₂ aldehydes and/or C₃₋₁₂ ketones can be determined by GC-MS. A suitable GC-MS measurement protocol is described as follows: The sample is analysed with combined GC Agilent 7890 and MS Agilent MSD 5975. For the GC, a capillary Wax column (for example Zebron™ ZB-Wax) (length=60 cm, diameter: 0.32 mm, df=0.5 μm, wherein df=film thickness), flow 1.2 mL/min is used, running with a temperature gradient of 40° C. (6 min)->10° C./min->250° C. As standard, a solution of target impurities in acetic acid is used, also for column calibration.

In the present invention, “ketene” denotes substances of the structure R¹R²C═C═O, wherein R¹ and R² independently can be hydrogen, alkyl or alkylene which can optionally be branched and/or substituted. In particular, either R¹ and R² or both can be the same as R³, aryl, acyl and halogen.

According to a most preferred embodiment of the present invention, the term “ketene” denotes the compound of the formula H2C═C═O, wherein R¹ and R² in the above R¹R²C═C═O are H.

Carboxylic anhydrides, according to the present invention, denote compounds consisting of two acyl groups bonded to the same oxygen atom, which can be denoted as R¹R²HC—C(O)—O—C(O)—R³, wherein R³, R¹ and R² are the same as described above. In the present invention, R¹ and R² preferably are H, straight or branched C1 to C6 alkyl, in particular linear C1-C4 alkyl or branched C3-C6 alkyl, or unsaturated C3-C6 alkyl, wherein alkyl can optionally be substituted by at least one substituent selected from the group consisting of halogen atoms, in particular fluorine, OH and CN. Acetic anhydride, wherein both R¹ and R² are H, is preferred according to the present invention.

The carboxylic acid can also be a mixture of two or more carboxylic acids. In this case, firstly asymmetrical carboxylic anhydrides can form in the process according to the present invention. Secondly, different ketene species can form when a mixture of two or more carboxylic acids are subjected to thermal treatment, in particular cracking. In another aspect, asymmetric carboxylic anhydrides are formed if R¹R²C═C═O is reacted with R³COOH, when R¹R²HC≠R³.

In one embodiment of the invention, the ketene is produced or generated by thermal cracking of a carboxylic acid, in particular acetic acid, as the principal ingredient, wherein the carboxylic acid further comprises C₂₋₁₂ aldehydes and/or C₃₋₁₂ ketones. This embodiment corresponds to condition II as noted above. The basic technique of thermal cracking of carboxylic acid, in particular acetic acid, is known in the art, as described in “Ketenes, Ketene Dimers, and Related Substances”, Taeschler, C, Kirk-Othmer Encyclopedia of Chemical Technology, p. 1-54, 2010, whose relevant contents are incorporated by reference into the present patent application. Taeschler recites that for the thermal cracking high quality acetic acid is evaporated and cracked, while according to the inventive process C₂₋₁₂ aldehydes and/or C₃₋₁₂ ketones are present in the ketene production step.

In a preferred embodiment of the invention, to produce ketene the carboxylic acid and in particular the acetic acid containing C₂₋₁₂ aldehydes and/or C₃₋₁₂ ketones is heated in a suitable cracking furnace in the presence of a catalyst, such as triethyl phosphate. The cracking temperature often is from 500° C. to 800° C., preferably from 720° C. to 780° C. The cracking furnace is made of suitable, heat resistant material, for example Sicromal comprising 25% Cr, 20% Ni, and 2% Si. Often, the formed ketene stream is treated with a neutralization agent such as ammonia to neutralize the catalyst. In a preferred aspect of this embodiment, the ketene stream is first cooled to less than 100° C. before being fed to the subsequent reaction step, which is preferentially a reaction with a carboxylic acid. This makes removal of water, unconverted carboxylic acid and other by-products possible, which are condensed below 100° C. while ketene remains in gas form. In a preferred aspect of the present invention, the ketene is formed in a first reaction zone, which preferably has a temperature of from 500° C. to 800° C., preferably from 720° C. to 780° C., and then the ketene is reacted with a carboxylic acid to form carboxylic anhydride in a second reaction zone. Generally, the temperature of the second reaction zone is from 10° C. to 100° C. In another aspect of the invention, the ketene is reacted with carboxylic acid to form carboxylic anhydride in the same reaction zone.

In this embodiment, the carboxylic acid comprising C₂₋₁₂ aldehydes and/or C₃₋₁₂ ketones which is used to generate ketene generally contains from equal to or less than 3000 ppm by weight of C₂₋₁₂ aldehydes and/or C₃₋₁₂ ketones. Preferably, the content of C₂₋₁₂ aldehydes and/or C₃₋₁₂ ketones in the carboxylic acid is equal to or lower than 1500 ppm by weight, more preferably equal to or lower than 1300 ppm by weight, even more preferably equal to or lower than 1000 ppm by weight and most preferably equal to or lower than 700 ppm by weight. The amount of impurities can be determined by GC-MS, by the method described above. In another aspect of this embodiment, a content of C₂₋₁₂ aldehydes and/or C₃₋₁₂ ketones in the carboxylic acid from which ketene is formed by thermal treatment of equal to or more than 10 ppm, or even equal to or more than 20 ppm is tolerated.

If the ketene is manufactured by thermal cracking of a carboxylic acid comprising C₂₋₁₂ aldehydes and/or C₃₋₁₂ ketones, the carboxylic acid can be supplied to the thermal cracking step directly from the process in which the carboxylic acid comprising C₂₋₁₂ aldehydes and/or C₃₋₁₂ ketones is generated, in particular from a wood acetylation process. In one aspect, which is preferred, the carboxylic acid comprising C₂₋₁₂ aldehydes and/or C₃₋₁₂ ketones is submitted to at least one intermediate treatment step, in particular a purification step, prior to the cracking process. This purification step, as noted above, may slightly reduce the content C₂₋₁₂ aldehydes and/or C₃₋₁₂ ketones, but does not eliminate these impurities.

In case of an inventive process, where condition I is met, the ketene which is fed to the process for producing a carboxylic anhydride can be manufactured by thermal cracking of a carboxylic acid, which is free or substantially free of C₂₋₁₂ aldehydes and/or C₃₋₁₂ ketones, wherein “substantially free” denotes a content of C₂₋₁₂ aldehydes and/or C₃₋₁₂ ketones in the carboxylic acid of equal to or less than 9 ppm by weight, preferably equal to or less than 7 ppm by weight, and even more preferably equal to or less than 5 ppm by weight. In another embodiment, the carboxylic acid contains equal to or less than 1000 ppm by weight, preferably equal to or less than 500 ppm by weight and most preferably equal to or less than 300 ppm by weight of other impurities. The amount of C₂₋₁₂ aldehydes and/or C₃₋₁₂ ketones and other impurities can be determined by GC-MS, by the method described above.

In a further embodiment of the present invention, the ketene which is fed to the process for manufacturing a carboxylic anhydride is produced by other ketene manufacturing processes, for example reaction of the corresponding carboxylic anhydride bearing at least one hydrogen atom at position a with a base, or by Wolff rearrangement of a-diazoketones. In this case, carboxylic acid comprising C₂₋₁₂ aldehydes and/or C₃₋₁₂ ketones is used as an absorption acid for the ketene.

In another embodiment, the carboxylic acid comprising C₂₋₁₂ aldehydes and/or C₃-12 ketones is reacted with ketene to form a carboxylic anhydride. This embodiment corresponds to condition I. Often, the carboxylic acid comprising C₂-12 aldehydes and/or C₃₋₁₂ ketones is also denoted as “absorption acid”. In this embodiment, the carboxylic acid comprising C₂₋₁₂ aldehydes and/or C₃₋₁₂ ketones generally contains from 5 to 3000 ppm by weight C₂₋₁₂ aldehydes and/or C₃₋₁₂ ketones. Often, the content of C₂₋₁₂ aldehydes and/or C₃₋₁₂ ketones in the carboxylic acid is equal to or higher than 5 ppm by weight, preferably equal to or higher than 10 ppm by weight, more preferably equal to or higher than 15 ppm by weight and most preferably equal to or higher than 20 ppm by weight. Often, the content of C₂₋₁₂ aldehydes and/or C₃₋₁₂ ketones in the carboxylic acid is equal to or lower than 3000 ppm by weight, preferably equal to or lower than 1500 ppm by weight, more preferably equal to or lower than 1300 ppm by weight, even more preferably equal to or lower than 1000 ppm by weight and most preferably equal to or lower than 700 ppm by weight. In a preferred aspect, the content of C₂₋₁₂ aldehydes and/or C₃₋₁₂ ketones in the carboxylic acid which is fed to the process as absorption acid is from 10 to 100 ppm, more preferably from 10 to 50 ppm. The amount of impurities can be determined by GC-MS, by the method described above.

In case of an inventive process, where condition II is met, the carboxylic acid which is fed to the process of the present invention as absorption acid to react with ketene can be free or substantially free of C₂₋₁₂ aldehydes and/or C₃₋₁₂ ketones, wherein “substantially free” denotes a content of C₂₋₁₂ aldehydes and/or C₃₋₁₂ ketones in the carboxylic acid of equal to or less than 9 ppm by weight, preferably equal to or less than 7 ppm by weight, and even more preferably equal to or less than 5 ppm by weight. In another aspect, the embodiment, the carboxylic acid contains equal to or less than 1000 ppm by weight, preferably equal to or less than 500 ppm by weight and most preferably equal to or less than 300 ppm by weight of other impurities. The amount of impurities can be determined by GC-MS, by the method described above.

The carboxylic acid comprising C₁₂ aldehydes and/or C₃₋₁₂ ketones which is reacted with ketene as absorption acid can be supplied to the reaction with ketene directly from the process in which the carboxylic acid comprising C₁₂ aldehydes and/or C₃₋₁₂ ketones is generated, in particular from a wood acetylation process. Preferably, however, the carboxylic acid comprising C₁₂ aldehydes and/or C₃₋₁₂ ketones is submitted to at least one intermediate treatment step, preferably a purification step, prior to the reaction with ketene. As noted above, this purification step may slightly reduce the content C₂₋₁₂ aldehydes and/or C₃₋₁₂ ketones, but does not eliminate these impurities.

The at least one purification step, to which the carboxylic acid is subjected either before being introduced in a ketene manufacturing unit or before being used as an absorption acid, is preferably selected from the group consisting of decantation, stripping, spinning, filtration, evaporation, condensation, steam treatment and treatment with an additive, for example an aqueous phase and/or a metal salt. In a preferred aspect of the invention the intermediate treatment of carboxylic acid comprising C₁₂ aldehydes and/or C₃₋₁₂ ketones is a purification which comprises
a) treating the carboxylic acid comprising C₁₂ aldehydes and/or C₃₋₁₂ ketones with a metal salt in the presence of water
b) treating at least part of the composition obtained in step a) to remove metal salt and optionally
c) subjecting at least part of the composition obtained in step b) by at least one operation selected from the group of operations consisting of distillation, stripping or membrane separation to recover at least a purified carboxylic acid fraction.

The metal salt often is a basic metal salt, such as alkaline earth acetate or hydroxide or alkaline acetate or hydroxide. The preferred metal salt in step b) is sodium acetate and/or sodium hydroxide. By means of the introduction of such salts, halogenides are converted to corresponding halogenide salts, and the corrosiveness of the carboxylic acid is thus reduced.

In one aspect of the invention, the carboxylic acid comprising C₁₂ aldehydes and/or C₃₋₁₂ ketones is supplied both to the cracking step to form ketene and as absorption acid in the reaction step where the ketene is reacted with the carboxylic acid to form the carboxylic anhydride. In other words, conditions I. and II. are met simultaneously, wherein in condition I., at least part of the carboxylic acid, which is reacted with ketene to produce a carboxylic anhydride, is a carboxylic acid comprising C₁₂ aldehydes and/or C₃₋₁₂ ketones, in particular a carboxylic acid comprising from 5 to 3000 ppm by weight, more particularly from 5 to 1500 ppm, most particularly from 10 to 100 ppm by weight of C₁₂ aldehydes and/or C₃₋₁₂ ketones, and wherein in condition II. at least part of the ketene is manufactured from a carboxylic acid, which comprises C₁₂ aldehydes and/or C₃-12 ketones, in particular equal to or less 3000 ppm, more particularly 1500 ppm by weight of C₁₂ aldehydes and/or C₃₋₁₂ ketones, and wherein the ketene is manufactured by a thermal treatment process. In one aspect of condition II., a content of comprising C₁₂ aldehydes and/or C₃₋₁₂ ketones of from 10 to 100 ppm by weight, is most preferred. The carboxylic acid comprising C₁₂ aldehydes and/or C₃₋₁₂ ketones independently in condition I. and II. can be used directly from their originating process, in particular wood acetylation process, or can be submitted to one or more purifications steps such as described above.

In a yet further embodiment the inventive process further comprises at least one purification step, to which the carboxylic anhydride produced in the process is subjected. In other words, after the carboxylic anhydride has been generated in the process by combination of absorption acid and ketene, it is subjected to at least one further purification. Preferred as such purification step is a purification, wherein compounds with a higher and lower boiling than the carboxylic anhydride are removed. A particularly preferred purification of this type is rectification. Most preferred is a two step rectification, wherein in a first step the carboxylic anhydride is obtained as the bottom product (whereas contaminants with a lower boiling point are removed) and wherein in the second rectification the carboxylic anhydride is removed as the top product (whereas contaminants with a higher boiling point are removed). By means of such purification, it can suitably be ensured that in a process where a regenerated carboxylic anhydride is continually used in a wood treatment process, contaminants, which are eluted from the wood during the treatment are not accumulated and that also by-products formed in the carboxylic anhydride production process are removed. As this is done directly before the carboxylic acid is again reacted with polysaccharides or wood, impurities are removed in one step, as opposed to a thorough purification of the carboxylic acid after wood treatment and of the carboxylic anhydride after regeneration from the carboxylic acid.

The process according to the invention and its specific aspects and embodiments of the present invention can suitably be run batch-wise or continuously, and/or can be part of another batch-wise or continuous process comprising further steps. Preferably, the process is a continuous process.

The invention further concerns a process for the manufacture of treated wood, in particular acetylated wood, comprising the process for the manufacture of a carboxylic anhydride according to the foregoing embodiments.

The invention also concerns the use of carboxylic anhydride, in particular acetic anhydride, which is obtained by the process according to the foregoing embodiments, in a process for acylation of polysaccharides. Preferably, the process for acylation of polysaccharides is a wood acetylation process.

Should the disclosure of patents, patent applications and publications which are incorporated herein by reference conflict with the description of the present application to the extent that it may render a term unclear, the present description shall take precedence.

Should the disclosure of any patents, patent applications, and publications which are incorporated herein by reference conflict with the description of the present application to the extent that it may render a term unclear, the present description shall take precedence.

The examples which follow are intended to illustrate the present invention without, however, limiting the scope thereof.

### Example

Acetic acid, which is produced as effluent stream in a wood acetylation process (content of C₂₋₁₂ aldehydes and/or C₃₋₁₂ ketones of 55 ppm), is filtered and distilled to yield a purified acetic acid with a content of C₁₂ aldehydes and/or C₃₋₁₂ ketones of about 40 ppm by weight. The purified acetic acid is reacted as absorption acid with ketene, which is produced in a first reaction zone at about 730° C. from acetic acid in the presence of triethyl phosphate, in a second reaction zone at a temperature of 25 to 60° C. to yield acetic anhydride.

1. Process for the production of a carboxylic anhydride, wherein a carboxylic acid is reacted with a ketene to produce the carboxylic anhydride, wherein at least one of the following conditions is met:
I. the ketene is introduced into a carboxylic acid comprising C₂₋₁₂ aldehydes and/or C₃₋₁₂ ketones to thus form carboxylic anhydride and/or
II. at least part of the ketene is manufactured from a carboxylic acid comprising C₂₋₁₂ aldehydes and/or C₃₋₁₂ ketones, and the thus produced ketene is reacted with a carboxylic acid to thus form carboxylic anhydride.

2. Process according to claim 1,
wherein the carboxylic acid in condition I. comprises from 5 to 3000 ppm by weight of C₂₋₁₂ aldehydes and/or C₃₋₁₂ ketones, and/or wherein the carboxylic acid in condition II. comprises equal to or less than 3000 ppm by weight of C₂₋₁₂ aldehydes and/or C₃₋₁₂ ketones.

3. Process according to claim 1 or 2,
wherein the carboxylic anhydride is acetic anhydride.

4. Process according to anyone of claims 1 to 3,
wherein carboxylic acid comprising C₂₋₁₂ aldehydes and/or C₃₋₁₂ ketones originates from a wood treatment process, preferably from a wood acetylation process.

5. Process according to anyone of claims 1 to 4,
wherein the carboxylic acid is selected from the group consisting of acetic acid, trifluoroacetic acid, propionic acid, n-butyric acid, iso-butyric acid and acrylic acid, preferably wherein the carboxylic acid is acetic acid.

6. Process according to anyone of claims 1 to 5,
wherein the carboxylic anhydride is subjected to at least one purification step to provide a purified carboxylic anhydride, wherein the purification is preferably rectification.

7. Process according to any one of claims 1 to 6,
wherein condition II. is met, and wherein the ketene is produced by a thermal treatment process.

8. Process according to any one of claims 1 to 7,
wherein the ketene is manufactured in a first reaction zone, and the reaction of ketene with carboxylic acid is performed in a second reaction zone.

9. Process according to claim 8,
wherein the first reaction zone has a temperature of from 500° C. to 800° C., preferably from 720° C. to 780° C., and wherein the second reaction zone has a temperature of from 10° C. to 100° C.

10. Process according to any one of claims 1 to 9,
wherein the carboxylic acid comprising C₂₋₁₂ aldehydes and/or C₃₋₁₂ ketones is provided to the process for manufacturing carboxylic anhydride without intermediate treatment, in particular without purification.

11. Process according to any one of claims 1 to 9,
wherein the carboxylic acid comprising C₂₋₁₂ aldehydes and/or C₃₋₁₂ ketones is provided to the process for manufacturing carboxylic anhydride with intermediate treatment, in particular with one or more purification steps selected from the group of steps consisting of evaporation, stripping, filtration, steam treatment, addition of additives and condensation.

12. Process according to claim 11,
wherein the intermediate treatment of the carboxylic acid comprising C₂₋₁₂ aldehydes and/or C₃₋₁₂ ketones is a purification which comprises
a) treating the carboxylic acid comprising C₂₋₁₂ aldehydes and/or C₃₋₁₂ ketones with a metal salt in the presence of water
b) treating at least part of the composition obtained in step a) to remove metal salt and optionally
c) subjecting at least part of the composition obtained in step b) to at least one operation selected from the group of operations consisting of distillation, stripping or membrane separation to recover at least a purified carboxylic acid fraction.

13. Process for the manufacture of treated wood, in particular acetylated wood, wherein the process comprises a process for the production of a carboxylic anhydride according to anyone of claims 1 to 13 and a step of treating wood with the carboxylic anhydride thus prepared.

14. Use of carboxylic anhydride, in particular acetic anhydride, which is obtained by the process according to anyone of claims 1 to 12, in a process for acylation of polysaccharides.

15. Use according to claim 14,
wherein the process for acylation of polysaccharides is a wood acetylation process.
